(19) United States
(12) Patent Application Publication
(10) Pub. No.: US 2008/0003326 A1
(43) Pub. Date: Jan. 3, 2008

(54) Enhanced Aquaculture Feeds

(75) Inventors: Peter James William Olive, Tyne and Wear (GB); Stephen Craig, Newcastle Upon Tyne (GB); Graham Rutherford, North Shields (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Seabait Limited, Ashington, Northumberland (GB)

(21) Appl. No.: 11/660,129

(22) PCT Filed: Aug. 5, 2005
(86) PCT No.: PCT/GB05/03104

§ 371 Date: Apr. 5, 2007

(30) Foreign Application Priority Data
Aug. 12, 2004 (GB) 0417977.6

(51) Int. Cl. A01K 67/033; A23K 1/18

(52) U.S. Cl. 426/2; 426/645; 426/72; 426/89; 426/92; 800/8

(57) ABSTRACT

There is provided a method of improving the nutritional content of marine worms, such as polychaetes, by feeding the worms a diet having a concentration of pigments, polyunsaturated fatty acids, lipids, vitamins and/or minerals sufficient to enhance the level of such components within the tissue of the worms. The worms can then be used for aquaculture, for example in farming marine fish and/or shrimps. One component of particular benefit is astaxanthin, which is preferably present in the polychaete diet of the worms at a concentration of at least 200 ppm. Advantageously the diet fed to the worms will include at least 10% by weight of vegetate oil. Conveniently the worms may be dried by lyophilisation or by refractance window drying before optionally being included or formed into pellets for aquaculture or other (i.e. aquarium) use.

The present invention relates to the enhancement of aquaculture feeds comprising marine worms.

Marine worms are animals in the Class Polychaeta of the Phylum Annelida or in the Phylum Sipunculida or are other such animals as may be generally referred to as worms which may be used as bait by anglers. Such worms are also used as foodstuffs for fish, crustaceans and other organisms, for toxicity testing and for other scientific purposes.

Cultured and naturally occurring marine worms are included in various ways into the diets of farmed marine animals, including cultured fin fish species, crustacean species and cultured polychaetes and could be included in the diets of any carnivorous or omnivorous marine species and in the feeds for terrestrial animals. They may be fed in live form as is common in many tropical aquaculture industries, supplied in a blast frozen form as whole polychaetes either singly or as a block, or incorporated into extruded formulated diets as specified by Cowboy feeds www.parkerintl.com and as proposed by Olive, 1999, Hydrobiological, 402: 175-183.

Cultured and naturally occurring polychaetes also are beneficial in the diets of many ornamental fish and animals living in marine and fresh water aquaria.

Naturally occurring supplies of marine worms are not inexhaustible, and collection of marine worms from the wild has been recognised as a cause of serious environmental concern.

Aquaculture of marine worms provides a sustainable source.

In nature, there are a number of polychaete worms, which have attracted the attention of the aquaculture industry and sea anglers. Among these, the Euncida and members of the Nereididae (ragworms), the Arenicolidae (lugworms) are particularly important (Gambi et al, 1994 Memoires de la Musee nationale d'Histoire naturelle, 162:593-603; Olive, 1993 Aquatic Conservation: Marine and Freshwater Ecosystems, 3(1):1-24). At the same time there have been concerns that bait digging for these animals may cause environmental damage (Olive, 1993, supra) and large-scale culture is now possible. The culture of these animals provide another source of polychaete materials for use in the aquaculture industry. Methods of enhancing the aquaculture of polychaete worms are described in WO-A-98/06255 and WO-A-98/44789. WO-A-98/06255 describes the use of cryopreservation techniques and also the manipulation of the photoperiod to control the time of sexual maturity of marine worms. WO-A-98/44789 describes controlling the photoperiod to enhance the growth of polychaete worms belonging to the Nereididae or Eunicidae families, typically the ragworm Nereis virens. A method for rearing Arenicola marina and Arenicola defodiens is described in WO-A-03/007701.

Marine worms typically comprise 80% water and hence are relatively expensive to transport. We have now recognised that reliable methods of concentrating the solid foodstuff without adversely affecting nutritional content would be of benefit to the aquaculture industry.

We have now found that frozen marine worms can be lyophilised without detriment to the nutritional content of the worms. The lyophilised worms can be transported and can optionally be subsequently re-hydrated before use. We have also found that the application of Refractance Window™ Drying (RWD) provides an alternative method of removing water from a tissue homogenate prepared from fresh or previously frozen and thawed samples of marine worms.

The present invention provides a method of processing marine worms, said method comprising drying the worms by RWD or by freeze-drying. By “freeze-drying” we refer to a process of freezing marine worms to obtain frozen marine worms and lyophilising the frozen marine worms.

The freezing step in the freeze-drying process may use any suitable method of freezing but desirably reduces the temperature of the marine worms to at least −5° C., preferably −10° C. or lower. A temperature of −20° C. may be required for certain embodiments.

In one embodiment the freezing step may be achieved with a blast freezer. Exemplary equipment includes the BF35 Cabinet Freezer (Foster).

The frozen worms can be subjected to a lyophilisation step immediately or can be stored in a suitable commercial or domestic freezer until required. Generally storage at −20° C. is not detrimental for a period of up to 7 months.

A number of commercially available lyophilisation equipment is available from, for example, Christ Freeze Dryers, Derbyshire, UK; LTE Lyo Trap Freeze Dryers, Oldham, UK; and ILMVAC, West Sussex, UK. The procedure involves the holding of previously frozen samples of biological material in reduced atmospheric pressure in a unit that typically also incorporates chilling and condenser units. The process of lyophilisation involves the sublimation of the ice in the samples at reduced pressure.

A 5 kg capacity machine (reduced temperature condenser −60° C. and atmosphere of 0.01 mbar) used for a period of 24 hours is sufficient to lyophilise for a sample of 5 kg (wet mass) of marine worms.

In one embodiment the lypophilised worms can be ground into a particulate matter prior to transportation, or thereafter.

The application of RWD technology can be achieved using commercially available equipment (eg. from Desert Lake Technology LLC or MCD Technologies, Inc of Tacoma Wash., USA). In essence, RWD uses heated water to dry raw material lying on a clear plastic membrane on the water surface. Heat energy is only passed through the membrane whilst the material is wet, when the membrane acts as a “refractance window”. Dry material is protected from the heat due to the poor heat conduction of the plastics membrane.

In RWD, heat is typically applied to the material at 72° C. for three to five minutes which enables excellent preservation of nutrient content.

In the present invention, worm material biomass is homogenised, optionally with the addition of water, to produce a slurry of the required consistency. In one embodiment the jaws of the worm are homogenised further to provide a significant source of zinc which is a valuable component for aquaculture.

Any suitable homogeniser or liquidiser can be used. We have found that up to 50% w/w water may be added to achieve a suitable viscosity for RWD. The water added may be fresh water or could include salt e.g. be saline.

The marine worms may be collected from their natural habitat, but more preferably are cultured worms farmed specifically for use as a bait or feedstuff for other marine, brackish water or freshwater animals.

The marine worms are preferably polychaetes, for example Nereis virens, Pereinereis nuntiae or Arenicola marina.

In one embodiment the drying of the marine worms results in a moisture content of 10% or less. In one embodiment the moisture content of the dried worm material is 5% or less.

In one embodiment lyophilisation results in a percentage reduction in mass of 75% or greater. The percentage reduction in mass of the marine worms is calculated by measuring the wet mass of a sample pre and post-lyophilisation.

In one embodiment, RWD results in a percentage reduction in mass of 75% or greater (calculated as outlined above).

In one example of RWA a percentage reduction in mass was over 80%, with 10 Kg (221b) dry weight material being recovered from 53.5 Kg (1181b) of wet material applied.

In one embodiment the dried worms can be included or formed into pellets, crumb or flake for aquaculture or other (ie aquarium) use.

We have further found that the nutritional content of marine worms can be modified by manipulation of their diet during culture. Thus, the marine worms can be grown for use as a foodstuff for a specific pre-determined end organism, for example finfish, penaeids etc. Farmed salmon, sea bream, seabass, sole, many tropical fish species and brackish, fresh and marine shrimp are of interest.

In farmed marine organisms such as finfish and penaeids it is important to provide sufficient pigments, polyunsaturated fatty acids, lipids, vitamins and/or minerals in the diet.

Thus, the present invention provides a method of increasing the nutritional content of marine worms, wherein the marine worms are fed a diet having a concentration of pigments, polyunsaturated fatty acids, lipids, vitamins and/or minerals thereby enhancing the level of these components within the tissues of the worms.

Suitable pigments include astaxanthin and carotenoids, for example beta carotene.

In one embodiment the marine worms are fed a diet containing at least 200 ppm astaxanthin. The marine worms fed this diet would contain 10 to 30ppm of free, unbound astaxanthin.

In one embodiment, the astaxanthin content of the marine worms is enhanced by feeding the worms the red algae Haematococcus pluvialis.

In one embodiment, the marine worms are allowed to consume algae optionally containing astaxanthin growing on the substrate in which they are cultured. Generally the alga, which may contain astaxanthin as a constituent pigment, will be seeded or pre-cultured on the substrate.

In one embodiment the vitamins can be vitamin C and/or vitamin E.

In one embodiment the minerals can be manganese, iron, nickel, copper, zinc, barium, and/or selenium, or mixtures thereof. Mention may also be made to cobalt, lead, aluminium and gold.

In one embodiment, the lipid content of the marine worm is enhanced by feeding the worms a lipid-enriched diet. The lipid-enrichment may be achieved by use of a vegetable oil, for example rape seed oil in the foodstuff provided to the worms or as a supplement thereto. Other suitable vegetable oils include corn oil, palm oil, safflower oil, soya oil, sunflower oil, groundnut oil, cottonseed oil and cocoa butter. A mixture of such oils may also be used. Fatty acids of especial interest for assimilation in marine worms include arachidonic, docosahexaenoic, eicosapentaenoic and cis-vaccenic acids.

The present invention further provides the use of a Haematococcus pluvialis or vegetable oil to enhance the nutritional content of marine worms.

The marine worms may be collected from their natural habitat, but more preferably are cultured worms farmed specifically for use as a bait or feedstuff for other marine animals.

The marine worms are preferably polychaetes, for example Nereis virens, Perinereis nuntiae or Arenicola marina.

In a further aspect, the present invention provides an aquaculture pellet comprising a coating of vegetable oil, which is suitable for feeding to marine worms in accordance with the invention. The vegetable oil can be sprayed onto conventional pellets or the pellets can be soaked in the oil before use. Suitable vegetable oils are rape seed oil, corn oil, palm oil, safflower oil, soya oil, sunflower oil, ground nut oil, cottonseed oil, cocoa butter or a mixture thereof. Optionally, pigments, vitamins and/or minerals can be admixed with the oil before application to the pellets. we have found this approach to be a quick and efficient way of introducing Haematoccus pluvialis and minerals such as trace elements to the diet of marine worms.

In a further aspect, the present invention provides a marine worm containing at least 6% dry weight of polyunsaturated fatty acids. The reference to “percentage dry weight” is in respect to the dried biomass of the whole worm. Preferably a significant proportion of the polyunsaturated fatty acids is cis-vaccenic acid and in one embodiment the worm will contain 1.5% cis-vaccenic acid (by dry weight of biomass).

In a further aspect the present invention provides a marine worm containing at least 10 ppm astaxanthin (by dry weight of biomass). In one embodiment the marine worm contains up to 30 ppm astaxanthin (by dry weight of biomass).

Such worms are particularly useful for aquaculture feeds or for other uses (eg. aquarium feeds). The worms may also be processed into a dried (usually powdered or ground) material. Optionally the dried worm material can be further processed into pellet, crumb or flake for aquaculture, aquarium or other uses.

The present invention will now be further described with reference to the following, non-limiting examples and figures in which:

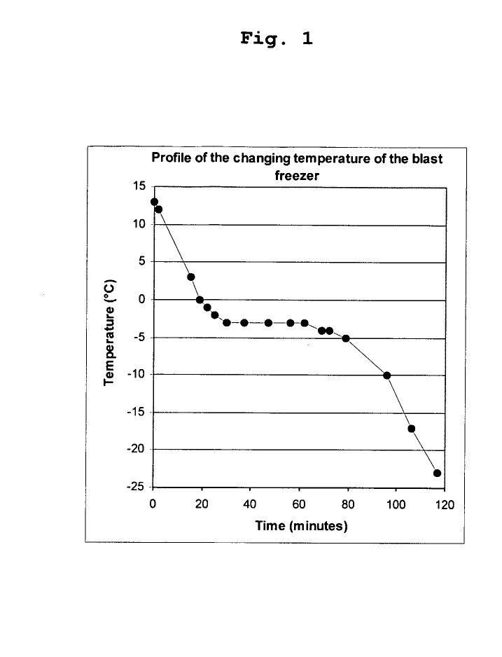

FIG. 1 shows the temperature profile of a typical freezing cycle of marine worms subjected to blast freezing.

### EXAMPLES

### Example 1

### Lyophilisation of Polychaete Biomass

Polychaete worms of the species Nereis virens were taken from culture tanks where they had been grown from larvae (see WO-A-98/06255 and WO-A-98/44789 for culture details). The worms were depurated by being held in a tank of clean flowing sea water in the absence of sediment, the dimensions of the tank being 1 metre by 5 metres with a depth of 15 cm of water. We have found that this dimension is suitable to allow quantities of several kilograms of worms to separate themselves from debris, sediment, faeces and other unwanted materials by virtue of their natural movements although other tank sizes can also be used. The worms were then removed from the depuration chamber by net and transferred to a grading table and any unwanted damaged or particulate materials were removed.

The worms were then packaged in sealed plastic bags. For convenience bags holding 450 g to 454 g of worms were used; though any suitable quantity, size or container may be selected.

The bags containing worms were then blast frozen using a commercially available equipment to minimise degradation of the chemical and biochemical components including elements known to be beneficial to other cultured species by virtue of the positive effects on breeding, sexual maturation or other life processes. The temperature profile of a typical freezing cycle is presented in FIG. 1.

The polychaete worms could then be stored in a commercial or domestic freezer maintaining the temperature at approximately −20° C. or lower prior to transfer to the lyophilisation chamber.

A number of 450 g to 454 g samples of blast frozen worm tissue were exposed by cutting away a portion of the plastic bag in which they were frozen and were then introduced to the lyophilisation chamber. More than one bag of frozen material could be lyophilised at one time depending on the specification of the equipment selected.

To determine the time required for lyophilisation samples may be taken from the chamber at various times during the process of sublimation and the mass recorded, until the moisture content is reduced to less than 5% as observed by the stabilisation of mass.

The percentage reduction in mass was 81.2±2.5% for more than 14 experimental samples. The samples incorporated a residual water content of 2.5%.

Material that has been frozen and lyophilised in this way may now be processed to produce materials suitable for individual users or market requirements.

### Example 2

### Analysis of Nutritional Content of Lyophilised Polychaeta.

The method described provides an example of the provision of feed to the polychaete Nereis virens and the consequent accumulation of polyunsaturated fatty acids deemed important to various feed sectors including the aquaculture industry.

The process of lyophilisation is used for the preservation of the species prior to the analysis of the lipid and fatty acid component determination. Larvae and juveniles of ragworm (Nereis virens) were produced on the Seabait Ltd site (Bed 16, 130 days old). Known densities (approximately 1600 worms·box⁻¹) of larval animals were introduced (‘thinned’) into trial boxes. Animals were allowed to ‘settle’ (construct burrows) in sand within trial boxes for 24 hours. Feed was given after this period. A feed was ground to a uniform and easily replicated size using a grinder with a particle size adapter depending on worm size. Feed was administered to all experimental beds at 1% of the biomass and increased daily depending on feeding status.

After 30 days core samples (12 cm diameter) were taken from each box and stored in individual white plastic containers filled with seawater. All worms were depurated in seawater for 12 hours as described in Example 1. After 12 hours wet mass (excess water removed) was recorded for individual worms from each of the boxes. Each sample of worms was sealed in plastic bags and blast frozen (−29° C.) and stored at −20° C. until lyophilised as described in Example 1.

The same procedure was carried out at 60 and 90 days.

Lyophilised samples from the feed trials were analysed for total lipid, total protein, total ash, total astaxanthin and total free astaxanthin at various times as described. The data for animals from the 90 day samples are shown in Table 1.

TABLE 1
Proximate analysis of Nereis virens samples
from different feeding regimes

| | | | | | Astaxanthin |
|---|---|---|---|---|---|
| | | | | Astaxanthin | post |
| | Protein | Lipid | Ash | pre- | hydrolysis |
| | % DW | % DW | % DW | hydrolysis | μg/g |
| NVC1 | 54.15 ± 1.56 | 20.86 ± 1.34 | 8.05 ± 0.20 | n/a | <1.0 |
| NVC2 | 55.16 ± 0.97 | 18.09 ± 2.10 | 6.50 ± 0.16 | n/a | <1.0 |
| NVC3 | 50.97 ± 0.77 | 17.04 ± 0.83 | 5.80 ± 0.13 | n/a | <1.0 |
| NVSFA1 | 54.82 ± 0.25 | 20.43 ± 1.32 | 6.53 ± 0.05 | n/a | <1.0 |
| NVSFA2 | 51.83 ± 0.76 | 20.76 ± 0.46 | 7.03 ± 0.05 | n/a | <1.0 |
| NVSFA3 | 50.45 ± 1.47 | 25.93 ± 0.91 | 7.97 ± 0.28 | n/a | <1.0 |
| NVSFB1 | 50.68 ± 0.27 | 20.42 ± 0.81 | 7.33 ± 0.26 | n/a | <1.0 |
| NVSFB2 | 50.08 ± 0.67 | 19.93 ± 0.49 | 6.98 ± 0.10 | n/a | <1.0 |
| NVSFB3 | 50.49 ± 0.11 | 19.05 ± 0.21 | 6.81 ± 0.08 | n/a | <1.0 |

[Key: ± = Standard deviation; DW = dry mass; n/a = Not detected; trace = <0.04]

In Table 1 NVC1, 2 and 3 refer to animals provided with the coarse feed for 90 days; NVSFA1, 2 and 3 refer to animals provided with coarse feed for 60 days and then superior feed for days 60 to 90; NVSFB1, 2 and 3 refer to animals given coarse feed for 80 days and then superior feed for days 80 to 90. All analyses were carried out in triplicate.

Details of the course feed and superior feeds used are given below:

TABLE 1a

Composition

| | | Superior |
|---|---|---|
| Component | Coarse Feed | Feed |
| Protein (%) | 36.0 | 45.0 |
| Lipid (%) | 7.0 | 26.0 |
| Fibre (%) | 6.0 | 1.0 |
| Ash (%) | 12.0 | 8.0 |
| Water (%) | na | na |
| Phosphorus (%) | 1.4 | 1.2 |
| Copper (mg · kg⁻¹) | 10 | 10 |
| Astaxanthin (mg · kg⁻¹) | — | 75 |
| Vitamin C (iu · kg⁻¹) | — | 1000 |
| Vitamin A (iu · kg⁻¹) | 15000 | 15000 |
| Vitamin E (iu · kg⁻¹) | 100 | 310 |
| Vitamin D3 (iu · kg⁻¹) | 1200 | 2000 |

### Example 3

### Enhancement of the pigment content of Nereis virens

To determine whether beneficial nutrient elements were preserved in lyophilised previously blast frozen samples, feeding trials using Nereis virens were carried out. Samples of Nereis virens were selected after approximately 3 months of culture when they had a mean weight of approximately 1 g and they were presented with a number of different diets in which a standard coarse food commercially available pellet was supplemented with different forms of astaxanthin and nutrients, the supplements included: the red algal meal Haematoccocus pluvialis, vitamin and pigment rich emulsions and a water-soluble form of astaxanthin, namely lucantin® pink.

These supplements were applied to coarse pellets via ‘top dressing’/coating. This was done by spraying the pellets with the test material from a hand operated spray such as may be used to water house plants, though any suitable spray device would suffice. The excess coating was allowed to be absorbed by the pellet. The specification of the coarse feeds is given in Table 2.

TABLE 2
Composition of coarse pellets used in
feeding trials

| | Composition |
|---|---|
| | (dry weight) |
| | Coarse |
| Supplier | Trouw |
| Component | pellet |
| Protein (%) | 36.0 |
| Lipid (%) | 7.0 |
| Fibre (%) | 6.0 |
| Ash (%) | 12.0 |
| Water (%) | na |
| Phosphorus (%) | 1.4 |
| Copper (mg · kg⁻¹) | 10 |
| Astaxanthin (mg · kg⁻¹) | — |
| Vitamin C (iu · kg⁻¹) | — |
| Vitamin A (iu · kg⁻¹) | 15000 |
| Vitamin E (iu · kg⁻¹) | 100 |
| Vitamin D3 (iu · kg⁻¹) | 1200 |

The following solutions were selected for use as enrichment supplements for the standard pellet: Haematococcus pluvialis (marine algae); Lucantin® Pink and compared with un-enriched pellets. Analysis of the enriched pellets revealed that the level of pigment had been increased by up to 200 ppm astaxanthin.

Known densities (1000 worms·box⁻¹) of juvenile animals (3.0 g) were introduced into trial boxes (0.8 m²). Animals were allowed to ‘settle’ (construct and establish burrows) in the sand as described in Example 2 within trial boxes for 48 hours. Standard feed was then provided daily for 4 days before enriched feeds were provided.

Feed was administered to all experiments at 20 g per day (based on previous feeding levels and adjusted daily depending on feeding behaviour). Worms were fed the feed twice daily with the specified feeds. All worms were removed from boxes on day 30 and three bags each containing 100 g were produced from each box for the purpose of triplicate analysis of each treatment. The worms were blast frozen and lyophilised as described in Example 1. The growth increment (g·worm⁻¹·day⁻¹) and total biomass of worms was determined for each treatment for the 30 days. The samples of worms from each box were analysed for protein, lipid, ash, bound and free astaxanthin and for vitamins A, C and E. The results are shown in Table 3.

TABLE 3
Proximate and pigment analysis of Nereis
virens samples from different feeding regimes

| | | | | Astaxanthin | Astaxanthin |
|---|---|---|---|---|---|
| | | | | pre- | post |
| | Protein | Lipid | Ash | hydrolysis | hydrolysis |
| | % DW | % DW | % DW | (μg/g) | (μg/g) |
| LUC | 56.58 ± 0.57 | 19.73 ± 0.88 | 9.05 ± 0.21 | 0 | 0 |
| HP | 50.81 ± 1.30 | 18.00 ± 2.66 | 8.25 ± 0.42 | 0 | 7.53 ± 0.46 |
| CF | 56.89 ± 0.21 | 10.30 ± 0.70 | 10.55 ± 0.08 | 0 | 0 |

LUC - Lucantin Pink;
HP—Haematococcus pluvialis;
CF - coarse feed with no supplement;
DW dry mass; mean of the three samples for each treatment.

The most notable and significant result was the definite retention of free astaxanthin by Nereis virens fed on the algal meal Haematococcus pluvialis. The very small variation between the samples analysed for astaxanthin (animals fed on HP) suggests that the retention of astaxanthin by the body is a ‘real’ result. The form of astaxanthin in the algal meal is predominantly in the esterified form although Nereis virens is storing the pigment in a free form.

### Example 4

### Procedures to Further Enhance the Lipid and Pigment Contents

An extension of the invention as illustrated in Example 3 was to further increase the lipid and pigment contents using a variety of different procedures. These procedures are illustrated by the following examples.

A commercially available and inexpensive vegetable oil, which in this example was rape seed oil, comprising a number of C18 fatty acids, was used as a vector to carry astaxanthin rich Haematococcus pluvialis onto the surface of the standard pellet. An amount of the vegetable oil and pigmented rich algal meal was combined and a known volume of this mixture was added to a sample of the coarse feed and mixed together till a homogenous state was achieved. The final concentration of the pigment in two embodiments of the invention was 100 and 200 ppm. The worms were fed with the oil/pigment enriched feed as in previous examples being fed twice daily at a dose of 20 g per day.

Feeding trials were set up using H. pluvials algal meal to confirm that this source of astaxanthin is retained in tissues by Nereis virens and determine the form and concentration it is retained. The feed was supplied in different forms and concentrations (i.e. semi-moist feed pellets).

Semi-moist feeds also facilitated the incorporation of a number of different components in a homogenous and easily supplied form. Semi-moist feeds for N. virens trials were formulated and produced in the laboratory at Seabait Ltd. using a Kenwood Chef food processor with a pasta maker attachment. The various feeds were formulated as given in Tables 4a and 4b.

Feeds were made up using an extra fine powder feed (ground standard ‘coarse’ feed pellet).

TABLE 4a
Feed formulations for Feed trial (30 day
duration)

| | Astaxanthin |
|---|---|
| | content from |
| | H. pluvialis |
| Code | (μg · g⁻¹) |
| CF | 0 |
| V.H.2 | 200 |
| H.1 | 100 |
| H.2 | 200 |
| S.H.1 | 100 |
| S.H.2 | 200 |

TABLE 4b
Feed formulations for Feed Trial (20 day
duration)

| | Astaxanthin |
|---|---|
| | content from |
| | H. pluvialis |
| Code | (μg · g⁻¹) |
| CF | 0 |
| H.1 | 100 |
| H.2 | 200 |
| V.H.1 | 200 |

Key: CF—coarse feed;
V—vegetable oil;
H—H. pluvialis;
S—Semi-moist;
μg · g⁻¹ - parts per million (ppm);
Carrageenan was added at 1% as the gel binder for the semi-moist feed; water was added at 25% of the total wet mass.

The fatty acid profile of immature N. virens fed on the diet CF (i.e non-enhanced diet) is shown in Table 4c.

TABLE 4c
Typical fatty acid methyl ester profile Nereis
virens fed on a commercial diet

| FAME | Proportion (%) |
|---|---|
| C15:0 | 0.2 |
| C16:0 | 22.2 |
| C16:1 (n − 9) | 1.2 |
| C16:1 (n − 7) | 3.9 |
| C18:0 | 3.8 |
| C18:1 (n − 11) | 4.9 |
| C18:1 (n − 9) | 1.6 |
| C18:1 (n − 7) | 9.1 |
| C18:2 (n − 6) | 1.2 |
| C18:3 (n − 3) | 0.5 |
| C20:0 | 2 |
| C20:1 (n − 11) | 2.8 |
| C20:1 (n − 9) | 3.5 |
| C20:1 (n − 7) | 0.5 |
| C20:2 (n − 9) | 2.6 |
| C20:2 (n − 6) | 8.7 |
| C20:4 (n − 6) | 0.5 |
| C20:5 (n − 3) | 12.8 |
| C22:0 | 1.2 |
| C22:1 (n − 11) | — |
| C22:1 (n − 9) | 1 |
| C22:2 (n − 6) | 6.6 |
| C22:6 (n − 3) | 4.6 |
| Unidentified | 4 |
| SSAT | 28.3 |
| SMUFA | 27.5 |
| SPUFA | 40.2 |
| Total FAME (mg · g⁻¹) | 121.2 |

Samples were taken and treated as in the previous examples, blast frozen, lyophilised and analysed.

TABLE 5a
Proximate analysis for Feed Trial (30 day
duration)

| | Protein | Lipid | Ash |
|---|---|---|---|
| Code | % DW | % DW | % DW |
| CF | 51.3 | 17.2 | 7.7 |
| V.H.2 | 51.5 | 19.4 | 7.8 |
| H.1 | 49.8 | 17.7 | 8.7 |
| H.2 | 45.0 | 14.6 | 5.3 |
| S.H.1 | 48.8 | 15.3 | 6.4 |
| S.H.2 | 47.7 | 15.8 | 6.9 |

TABLE 5b
Proximate analysis for Feed Trial (20 day
duration)

| | Protein | Lipid | Ash |
|---|---|---|---|
| Code | % DW | % DW | % DW |
| C | 52.8 | 15.2 | 7.4 |
| H.1 | 52.8 | 16.0 | 7.5 |
| H.2 | 51.1 | 15.1 | 7.4 |
| V.H.1 | 50.0 | 20.1 | 8.1 |

Note: All samples were the mean of three replicate analyses; standard deviations were less than 0.5% in all cases.

The data in Tables 5a and 5b demonstrate the efficacy of the feeding regime in increasing the lipid content of the worms in a way that is desirable for their incorporation in aquaculture diets and that the enriched nutritional content of the worms is preserved by the application and operation of the invention as described.

Of particular interest in Table Sa is the lipid content value of 19.4 recorded for animals fed diet VH2 and in Table 5b the lipid content value of 20.1 recorded for animals fed diet VH1.

The impact of the procedures described on the astaxanthin content of lyophilised enriched worm tissues is shown in Table 6.

TABLE 6

| | Astax. | Astax. |
|---|---|---|
| Trial | (esterified)ppm | (free) ppm |
| Coarse feed | n/d | 0 |
| Coarse feed; HP; | n/d | 0 |
| vegetable oil | | |
| Coarse feed; HP | n/d | 0 |
| Coarse feed; HP | n/d | 0 |
| SM - pellet; HP; | n/d | 0 |
| SM - pellet; HP; | n/d | 0 |
| SM - pellet; 100 ppm HP | n/d | 14.7 |
| Coarse feed; 100 ppm HP | n/d | 12.1 |
| Coarse feed; 200 ppm | n/d | 12.6 |
| HP; VO | | |
| SM - pellet; HP 200 ppm | n/d | 20.6 |

### Example 7

### The Enhancement of the Nutritional Composition of Lyophilised Polychaeta Specifically Arenicola sp. Fed on a Variety of Feed Products

The example describes the provision of feed in the form of brewery yeast or other suitable dietary components to the polychaetes Arenicola marina and Arenicola defodiens in a method described in WO-A-03/007701. Application of a suitable feed to the specified substrate results in the growth of Arenicola sp. (lugworms) and an increase in the levels of polyunsaturated fatty acids (PUFA) including cis-vaccenic, Arachidonic acid (AA), Eicosapentaenoic acid (EPA) and Docosahexaenoic Acid (DHA). These fatty acids are accumulated in the tissues of the polychaetes A. marina (commonly referred to as ‘blow lug’) and A. defodiens (commonly referred to as ‘black lug’, or ‘yellow tails’) even when the initial feed is devoid of these fatty acids. The fatty acid cis-vaccenic is a precursor to arthropod sex pheromones and plays a significant role in maturation of the gametes of important commercially cultured aquaculture species including finfish and penaeids. The fatty acid AA is a precursor for a number of leukotrienes and eicosanoids including prostaglandins such as PGF2α, which is considered important in the maturation of shrimp species including those applied to culture conditions, which includes the penaeids, for example the white shrimp Litopenaeus vannemei and the black tiger shrimp Penaeus monodon Dcroz et al., 1988. “Prostaglandins and related compounds from the polychaete worm Americonuphis reesei as possible inducers of gonadal maturation in Penaeid shrimps”. Revista de Biologia Tropical 36, 331-332). At a cellular level prostaglandins and eicosanoids play a role in the elaboration of physiological responses triggered by hormones and other signal molecules which may have a significant role in influencing the maturation of cultured shrimp. The fatty acids EPA and DHA are required by all animals for incorporation into membranes as phospholipids and for the production of eicosanoids (eg. prostaglandins, leukotrienes).

Feeding and Proximate Composition of Lugworm (Arenicola marina)

Feeding trials were constructed with juvenile Arenicola marina to determine the growth rate and accumulation of specific components (for example protein, lipid and ash) after feeding with different feed products. Juveniles Arenicola sp. were produced in accordance with WO-A-03/007701. All juveniles were initially held in a mini-recirculation unit then stocked into 22 m² concrete culture beds. The mean size of the worm at the start of the trials was 0.05 g.

Small trial boxes (0.3m²) some of which contained brewery yeast and the effluent from a recirculation fish farm mixed into the substrate as described in WO-A-03/007701. All boxes were supplied with ‘flow through’ of warm seawater water (16° C.±1.5° C.).

Juvenile A. marina were introduced into the boxes at 100 per box (approximately 300 worms·m⁻²). Worms were left for 90 days. At the end of the 90 days the worms were removed and growth rates and proximate composition after blast freezing and lyophilisation was determined. Lyophilisation was used to preserve animal tissue for subsequent analysis of protein, lipid and other biochemical components. The proximate analyses of Arenicola sp. are presented in Table 7.

TABLE 7
Proximate analysis of lyophilised Arenicola
marina that were provided with different feeds.

| | | | | | Astaxanthin |
|---|---|---|---|---|---|
| | | | | Astaxanthin | post |
| | Protein | Lipid | Ash | pre- | hydrolysis |
| Details | % DW | % DW | % DW | hydrolysis | (μg/g) |
| AM.J | 62.81 ± 2.00 | 11.26 ± 0.40 | 5.42 ± 0.23 | n/a | 0.02 ± 0.01 |
| A.AM.BY | 62.20 ± 0.60 | 13.70 ± 0.46 | 5.19 ± 0.13 | n/a | 0.01 ± 00 |
| A.AM.FE | 62.70 ± 1.20 | 13.92 ± 0.94 | 6.03 ± 0.25 | n/a | 0.01 ± 00 |

[KEY: ± = standard deviation; n/a = Not detected; A = Adult; J = juvenile; AM = Arenicola marina; NT = no treatment; BY = brewery yeast; FE = fish effluent; DW - dry mass]

Analysis of lyophilised gravid female and male Arenicola marina fed on brewery yeast was also carried out (Table 8).

There was no significant difference between the protein content of adult and juvenile A. marina. Protein levels of A. marina was higher than that of Nereis virens. The fatty acid profile of Arenicola marina fed on brewery yeast was very typical of marine animals indicated by the presence of all important fatty acids including DHA, EPA and AA (Table 9).

TABLE 8
Proximate analysis of lyophilised gravid male and female
Arenicola marina that had been fed on brewery yeast.

| | | | | | Astaxanthin |
|---|---|---|---|---|---|
| | | | | Astaxanthin | post |
| | Protein | Lipid | Ash | pre- | hydrolysis |
| | % DW | % DW | % DW | hydrolysis | μg/g |
| AM.F.GR | 57.25 ± 1.44 | 18.30 ± 1.01 | 7.80 ± 0.32 | 0 | 0 |
| AM.M.GR | 68.71 ± 0.35 | 18.20 ± 0.99 | 6.80 ± 0.09 | 0 | 0 |

Key:
AM—Arenicola marina;
F—female;
M—male;
GR—gravid
±—standard deviation generated from a mean of at least three analyses;
parts per million - μg/g.

There was a significantly higher lipid content of gravid (fully mature adults) Arenicola marina compared with immature and maturing animals.

The fatty acid profile of Arenicola marina fed the diet A.AM.BY (from animals of Table 7) is shown in Table 9.

TABLE 9
Fatty acid profile of Arenicola marina fed
on brewery yeast.

Fatty acid methyl ester
profile of A. marina fed
on brewery yeast
Average with SD

| FA | mg/g DW | SD | % | SD |
|---|---|---|---|---|
| C14:0 | 1.34 | 0.31 | 2.87 | 0.16 |
| C15:0 | 0.77 | 0.14 | 1.66 | 0.01 |
| iso 15:0 | 1.02 | 0.20 | 2.19 | 0.01 |
| C16:0 | 8.81 | 1.87 | 18.93 | 0.68 |
| C16:1n − 7 | 3.21 | 0.72 | 6.89 | 0.21 |
| C16:1n − 5 | 0.35 | 0.06 | 0.76 | 0.02 |
| iso 16:0 | 1.96 | 0.37 | 4.22 | 0.15 |
| anteiso 16:0 | 0.88 | 0.21 | 1.90 | 0.13 |
| anteiso 16:1n − 5 | 0.42 | 0.06 | 0.90 | 0.11 |
| C16:2n − 4 | 0.17 | 0.04 | 0.36 | 0.04 |
| C17:0 | 0.59 | 0.08 | 1.28 | 0.10 |
| C18:0 | 0.78 | 0.33 | 1.72 | 0.83 |
| C18:1n − 13 | 0.66 | 0.17 | 1.40 | 0.13 |
| C18:1n − 9 | 3.80 | 0.80 | 8.18 | 0.68 |
| C18:1n − 7 | 7.14 | 0.98 | 15.48 | 0.83 |
| C18:1n − 6 | 0.28 | 0.02 | 0.61 | 0.08 |
| C18:2n − 6 | 1.87 | 0.58 | 3.98 | 0.45 |
| C19:1n − 9 | 0.11 | 0.02 | 0.23 | 0.05 |
| C19:1n − 6 | 0.30 | 0.05 | 0.66 | 0.05 |
| C18:3n − 3 | 0.71 | 0.18 | 1.53 | 0.19 |
| C20:0 | 0.09 | 0.09 | 0.21 | 0.23 |
| C20:1n − 9 | 2.42 | 0.15 | 5.29 | 0.62 |
| C20:2n − 7 | 0.62 | 0.04 | 1.37 | 0.20 |
| C20:2n − 6 | 1.26 | 0.37 | 2.69 | 0.27 |
| C20:3n − 7 | 0.49 | 0.17 | 1.05 | 0.15 |
| C20:4n − 6 (ARA) | 1.19 | 0.38 | 2.53 | 0.31 |
| C20:3n − 3 | 0.13 | 0.04 | 0.28 | 0.04 |
| C20:4n − 3 | 0.23 | 0.08 | 0.48 | 0.07 |
| C20:5n − 3 (EPA) | 2.97 | 0.65 | 6.38 | 0.43 |
| C22:1n − 9 | 0.14 | 0.01 | 0.31 | 0.05 |
| C22:4n − 6 | 0.42 | 0.09 | 0.91 | 0.02 |
| C22:5n − 3 | 1.03 | 0.18 | 2.23 | 0.10 |
| C22:6n − 3 (DHA) | 0.24 | 0.06 | 0.52 | 0.08 |
| Total | 46.44 | 8.83 | 100.00 | 0.00 |

The levels of cis-vaccenic acid (C18: 1n7) were higher than those found in fish tissue or marine fish oil which is in a frequent component of aquaculture feeds.

### Example 8

### Procedures to Further Enhance Biochemical Components of Polychaete Tissues via Submersion of Polychaetes in Solutions and Particulates Enhanced with Important Dietary Components

The method describes the enrichment of cultured and/or wild polychaetes with different pigments, vitamins or micro-elements via coating and/or absorption with any suitable vitamin, pigment or trace element enhanced particulate matter and/or solution/emulsion prior to or post undergoing a drying process (including for example the methods of lyophilisation, spray drying or air drying).

Live, cultured, depurated (as described in Example 1) Nereis virens which had any excess of water removed were submerged in a seawater solution containing different quantities of the algal meal Haematococcus pluvialis for different time periods. Animals were removed and immediately blast-frozen. Blast freezing was followed by lyophilisation of all samples. Lyophilised samples were milled and then proximate analyses of the samples were then carried out including the analysis of protein, lipid, ash, carotenoid and astaxanthin. The results of the submersion trials are presented in Table 10.

TABLE 10
Proximate analysis of Nereis virens after
different submersion/mucosal-coating treatments

| | | | | | Astaxanthin |
|---|---|---|---|---|---|
| | | | | Carote- | post |
| | | | | noid | hydrolysis |
| Details | Protein | Lipid | Ash | (μg/g) | (μg/g) |
| HP.1 | 52.04 ± 1.61 | 21.46 | 9.76 ± 0.03 | >5300 | >746 |
| HP.2 | 50.07 ± 0.42 | 23.31 | 9.66 ± 0.07 | >3700 | >464 |
| HP.3 | 49.93 ± 0.45 | 22.87 | 10.37 ± 0.28 | >16 | 0 |
| HP.4 | 53.85 ± 0.67 | 20.87 | 9.62 ± 0.10 | >300 | >36 |
| HP.5 | 54.53 ± 0.50 | 19.51 | 8.19 ± 0.12 | >1000 | >200 |
| HP.6 | 56.92 ± 0.71 | 20.76 | 9.62 ± 0.01 | >225 | >37 |

Key:
± = Standard deviation;
HP—Haematococcus pluvialis;
parts per million - μg/g
The coating trials resulted in a significant elevation of the carotenoid and free astaxanthin content of the lyophilised material.

### Example 9

### The Enhancement of Polychaete Tissue with Microelements

The invention describes the methodology for the enrichment of minerals, trace elements and physiologically important metals via provision of enhanced feeds to polychaete species including Nereis virens and Arenicola sp. The polychaetes Nereis sp. and Arenicola sp. can be enhanced with specific trace elements including iron, zinc, copper and selenium via the provision of a number of different feeds.

The metal composition of the polychaetes Nereis virens and Arenicola marina were enhanced by the provision of feeds including fish feed and brewery yeast. Nereis virens juveniles were fed a high protein diet and adults a standard (coarse) feed. Arenicola marina was fed on brewery yeast. All animals were blast frozen and then lyophilised for the metal analysis.

The results from the trace metal analysis of juvenile and adult N. virens and A. marina are presented in Table 11.

TABLE 11
Trace metal analysis of juvenile and adult
Nereis virens and Arenicola marina

| ICP-MS | | | | | | | ppm (μg/g) | | | | | | | |
|---|---|---|---|---|---|---|---|---|---|---|---|---|---|---|
| | Mn | | Fe | | Co | | Ni | | Cu | | Zn | | Pb | |
| Samples | Manganese | | Iron | | Cobalt | | Nickel | | Copper | | Zinc | | Lead | |
| NV-J | 6.8 | (3.5) | 708.7 | (1.6) | 0.2 | (0.4) | 1.4 | (0.7) | 4.2 | (3.4) | 80.4 | (1.1) | 0.6 | (1.3) |
| NV-A | 6.0 | (1.0) | 495.1 | (0.8) | 0.2 | (4.3) | 1.5 | (2.8) | 9.7 | (0.2) | 127 | (1.3) | 0.5 | (0.4) |
| AM-J | 12.1 | (1.4) | 857.7 | (1.4) | 1.1 | (1.9) | 3.4 | (2.6) | 2.2 | (2.5) | 83.6 | (1.1) | 1.7 | (0.6) |
| AM-A | 10.2 | (2.2) | 570 | (0.2) | 0.8 | (2.9) | 1.7 | (3.8) | 7.3 | (2.2) | 77 | (2.1) | 1.1 | (1.9) |

ICP-OES ppm (μg/g)

| | Al | | Ba | | Au | | Se | |
|---|---|---|---|---|---|---|---|---|
| | Aluminium | | Barium | | Gold | | Selenium | |
| NV-J | 1.32 | (0.6) | <0.01 | (1.3) | <0.01 | (7.8) | <0.01 | (272) |
| NV-A | 0.03 | (0.5) | <0.01 | (0.8) | <0.01 | (10.6) | <0.01 | (659) |
| AM-J | 0.72 | (3.1) | <0.01 | (1.9) | 0.01 | (8.3) | 0.02 | (1.4) |
| AM-A | 0.33 | (2.5) | <0.01 | (0.9) | <0.01 | (9.1) | 0.01 | (176) |

Key:
(n) = % expected error = Instrument error;
NV-J: juvenile Nereis virens;
NV-A: Adult Nereis virens;
AM-J: Juvenile Arenicola marina;
AM-A: Adult Arenicola marina.
μg/g = ppm - parts per million.

### Example 10

Dried polychaete material was incorporated in whole or in part (for example freeze dried, Refractance Window™ dried, air dried or spray dried polychaete material be it specific segments of the body or heads) into a semi moist or dried feed pellet or similar pellet, flake or feed component suitable for use as feed for aquatic species including, for example, those species used in aquaculture and aquarium systems. The feed pellet formed can also incorporate additional components including vitamins, minerals and pigments. The food pellets may be used to incorporate freeze dried polychaete material into shrimp maturation diets.

In particular semi moist pellets incorporating lugworm may be used to feed Nereis virens. A number of different components may be incorporated into semi moist feeds in a homogenous and easily supplied form. Semi moist feeds for N.virens (NV) trials were formulated and produced in the laboratory at Seabait Ltd. using a Kenwood Chef food processor with a pasta maker attachment. The feed (LUG) incorporated lyophilised lugworm at a proportion of 20%. Feeds were made up using an extra fine powder feed (ground standard ‘coarse’ feed pellet). The standard pellet (coarse feed; (CF)) was given to worms in the control box.

Approximately 1600 worms were introduced (thinned) into a trial box (having a side-area of approximately 0.8m²) containing sand. Animals were allowed to ‘settle’ (construct burrows) in the sand for 48 hours. The different feeds were provided after this period using standard farm protocols.

TABLE 12
Summary of growth data generated

| | | mean wet | mean wet | Growth |
|---|---|---|---|---|
| | Total | mass (g) | mass (g) | Increment |
| | biomass · m⁻² | start | end | (g · worm⁻¹ · day⁻¹) |
| NV.CF | 1489.7 | 0.8 | 1.4 | 0.03 |
| NV.LUG | 2025.6 | 0.7 | 1.7 | 0.05 |

Key:
NV—N. virens;
CF - coarse feed with no supplement;
Lug - lugworm Arenicola marina added;
DW dry mass;
mean of three samples in each case.

The feeding response of worms fed semi-moist diets containing lugworm was more rapid than those fed standard ‘coarse feed’. The greatest growth increment (g·worm⁻¹·day⁻1) was observed in the boxes fed lugworm incorporated into the semi moist diet (Table 12).

There was no difference in the composition of the animals fed on lugworm incorporated into the feed (Table 13).

TABLE 13
Proximate analysis for Feed Trial 3

| | Protein | Lipid | Ash |
|---|---|---|---|
| | % DW | % DW | % DW |
| NV.CF | 52.5 | 17.4 | 7.5 |
| NV.LUG | 47.9 | 16.0 | 5.2 |

Key:
NV—N. virens;
CF—coarse feed with no supplement;
Lug—lugworm Arenicola marina added;
DW dry mass; mean of three samples in each case.

Dried polychaetes (for example freeze dried Nereis virens) may be incorporated into a variety of feed types including for example pellets, crumbs, microparticulates, liquids, gels or other prepared feeds for supply into a number of markets (for example the aquarium, aquaculture and angling markets).

### Example 11

### Use of Refractance Window™ Drying to Prepare Cultured Polychaete Material

Cultured polychaete material, in a fresh, frozen or other form is homogenised to a slurry of desirable consistency (for example by adding 50% water, depending on the requirements of the machine) using any homogeniser, liquidiser or other machinery. The homogenised material is contained within any non-reactive unit or vessel such that degradation is minimised and preferably kept at a low temperature. The slurry is mixed with a known volume of water, be it fresh, salt, saline or any suitable salinity to produce a viscosity suitable for the process of RWD. Additional dietary or beneficial components or natural sources containing, for example vitamin C, astaxanthin, beta carotene, vitamin E, selenium, eicosapentaenoic acid, docosahexanenoic acid, arachidonic acid, etc can be added to the slurry at this time or at the point of homogenisation. The slurry is placed into a hopper for delivery onto the conveyor belt. In short the homogenised slurry is dispensed or sprayed onto a moving conveyor belt. The conveyor belt, which consists of, for example a plastic sheet, moves over a water bath of approximately 70° C. or any temperature deemed acceptable for the process to function efficiently. The polychaete slurry conducts heat (allows for the passage of infrared energy) while it contains water which results in evaporation and facilitating the dehydration of the material. Evaporation ensures the temperature of the polychaete slurry remains below that of the water bath and allows for maximum retention of nutrients. As the polychaete slurry dries (water content decreases) the energy from the water bath is refracted back into the water (effectively by closure the ‘window’ of infrared energy, the low water content no longer conducts the energy). The loss of heat from evaporation ensures the polychaete material receives minimal heating and thus optimises retention of nutritional components. At completion of progression along the conveyor the material contains around 5% water. The material is then scraped into a collection vessel of any non reactive material. The material is collected as a flake although can be processed, for example by grinding or milling, to a size that is required.

1. A method to enhance the nutritional content level within the tissues of marine worms, wherein the marine worms are fed a diet having a concentration of vegetable oil.

2. The method as claimed in claim 1 wherein the concentration of vegetable oil in the diet is sufficient to produce a level of polyunsaturated fatty acids of 6% dry weight.

3. The method as claimed in claim 1 wherein the marine worms are fed a diet including the pigment astaxanthin.

4. The method as claimed in claim 1 wherein the marine worms are fed a diet containing Haematococcus pluvialis.

5. The method as claimed in claim 1 wherein the marine worms are fed a diet containing at least 200 ppm astaxanthin.

6. The method as claimed in claim 1 wherein the marine worms are fed a diet containing at least 10% by weight of vegetable oil.

7. The method as claimed in claim 6 wherein said vegetable oil is rape seed oil, corn oil, palm oil, safflower oil, soya oil, sunflower oil, ground nut oil, cottonseed oil, cocoa butter or a mixture thereof.

8. The method as claimed in claim 1 wherein said marine worms are polychaetes.

9. The method as claimed in claim 8 wherein said marine worms belong to the family Nerididae.

10. The method as claimed in claim 9 wherein said marine worms are Nereis virens.

11. The method as claimed in claim 8 wherein said marine worms belong to the family Arenicolidae.

12. The method as claimed in claim 11 wherein said marine worms are Arenicola marina.

13. The method as claimed in claim 1 wherein said worms are also fed pigments, vitamins and/or minerals.

14. The method as claimed in claim 13 wherein said worms are fed a diet having iron, nickel, copper, zinc, barium and/or selenium or mixtures thereof.

15. An aquaculture pellet for feeding to marine worms, said pellet comprising a coating of vegetable oil.

16. A pellet as claimed in claim 15 wherein said vegetable oil is rape seed oil, corn oil, palm oil, safflower oil, soya oil, sunflower oil, ground nut oil, cottonseed oil, cocoa butter or a mixture thereof.

17. A pellet as claimed in claim 15 wherein said vegetable oil is admixed with Haematococcus pluvialis.

18. A pellet as claimed in claim 15 wherein said vegetable oil is admixed with at least one vitamin or mineral.

19. A pellet as claimed in claim 18 wherein said mineral is chosen from at least one of manganese, iron, nickel, copper, zinc, barium and selenium.

20. A marine worm containing at least 6% dry weight of polyunsaturated fatty acids obtainable from the method of any one of claim 1.

21. A marine worm as claimed in claim 20 containing at least 1.5% dry weight of cis-vaccenic acid.

22. A marine worm as claimed in 20 containing at least 0.2% dry weight of arachidonic acid.

23. A marine worm containing at least 10 ppm dry weight of astaxanthin obtainable from the method of claim 5.

24. (canceled)

25. (canceled)

26. A powdered or ground material comprising a marine worm cultured as claimed in claim 1.

27. A method of processing marine worms, said method comprising drying said worm either by i) freezing the worms and lyophilizing the frozen worms, or by ii) refractance window drying.

28. The method as claimed in claim 27 wherein said worms are frozen to a temperature of −5° C. or lower prior to lyophilisation.

29. The method as claimed in claim 27 wherein said worms are homogenized prior to refractance window drying.

30. The method as claimed in claim 27 wherein the worms have been fed a diet having a concentration of vegetable oil.
